(19) United States
(12) Patent Application Publication
(10) Pub. No.: US 2025/0011858 A1
(43) Pub. Date: Jan. 9, 2025

(54) WHOLE GENOME CPG ANALYSIS

(71) Applicant: NUCLEIX LTD., Rehovot (IL)

(72) Inventors: Danny FRUMKIN, Rehovot (IL); Adam WASSERSTROM, Ness Ziona (IL); Nimrod AXELRAD, Rehovot (IL); Revital KNIRSH, Rosh HaAyin (IL)

(73) Assignee: NUCLEIX LTD., Rehovot (IL)

(21) Appl. No.: 18/711,585

(22) PCT Filed: Nov. 17, 2022
(86) PCT No.: PCT/IL2022/051227

§ 371 Date: May 18, 2024

(30) Foreign Application Priority Data
Nov. 18, 2021 (WO) PCT/IL2021/051382
May 26, 2022 (GB) 2207784.6

(51) Int. Cl. C12Q 1/6869; C12Q 1/686

(52) U.S. Cl. CPC C12Q 1/6869; C12Q 1/686; C12Q 2600/154

(57) ABSTRACT

The invention provides methods for ultra-deep whole-genome sequencing of human DNA. The methods determine the methylation status of millions of sites in the human genome, wherein said methods use few (if any) DNA amplification cycles.

### FIELD OF THE INVENTION

This invention is in the field of epigenetic profiling of human DNA.

### BACKGROUND

Epigenetic modifications are an important regulator of gene transcription and genome stability. One of the key epigenetic modifications is methylation of DNA. This typically occurs at cytosine residues within ‘CpG sites’, which have the dinucleotide sequence CG. The methylation takes the form of conversion of the cytosines in the CpG site to 5-methylcytosine (5mC). 5mC may also undergo oxidation to 5-hydroxymethylcytosine (5hmC).

An insight into CpG methylation of cellular DNA can be obtained by analysing cell-free DNA (‘cfDNA’) which is released into blood. Gaining such an insight may be useful for the detection of cancer, as changes in CpG methylation are known to occur in diseases such as cancer.

Several techniques have been developed for the detection of DNA methylation, but all have drawbacks, especially for genomic analysis with low quantities of sample DNA (as with cfDNA). The gold standard for mapping DNA methylation is bisulfite sequencing, also known as bisulfite conversion sequencing. This is based on sodium bisulfite treatment of DNA to convert unmethylated cytosine to uracil. Differences in sequence between treated and untreated DNA permits methylation to be detected.

However, sodium bisulfite treatment causes extensive DNA degradation, so reducing the sensitivity of methylation analysis techniques based on this treatment. In part, this is because bisulfite conversion requires high temperatures and extreme pH. Data obtained by bisulfite sequencing is also biased, because unmethylated DNA is disproportionately damaged and bisulfite-treated DNA is more susceptible to amplification biases because of its reduced sequence complexity (a 4-base genome is reduced to a roughly a 3-base genome). In addition, the data is relatively noisy because of non-specific or incomplete conversion of bases and the above-mentioned reduced sequence complexity. As a result, bisulfite-derived libraries do not adequately cover the entire genome and include many gaps that are only sparsely covered, if at all. Critically, the high level of noise inherent to bisulfite sequencing means that it is poorly able to distinguish signal from noise when used to analyse cfDNA from blood for methylation changes from cancer cells. It is also cumbersome to carry out. Therefore, despite its popularity, using bisulfite conversion (coupled with next-generation sequencing, NGS) to analyse genomic methylation patterns at a single nucleotide level is a poor technique.

Other techniques for the detection of methylated DNA molecules, such as TET-assisted pyridine borane sequencing (TAPS, Liu et al., 2019) and EM-SEQ (Vaisvila et al., 2022), have similar problems. Both TAPS and EM-SEQ are associated with noise from false methylation and false unmethylation. They also involve a loss of sequence complexity, so any PCR performed during sequencing library preparation will suffer from the same issues as in bisulfite sequencing.

Thus, there is a need in the field for improved methods for analysing methylation patterns across the human genome.

### DISCLOSURE OF THE INVENTION

The inventor has found that it is possible to achieve high coverage of millions of CpG sites in the human genome with relatively little (if any) amplification of sample DNA, and the invention provides methods for whole-genome sequencing, wherein (i) human cfDNA from ≤10 ml blood is subjected to high throughput sequencing with an average depth of ≥600 across the genome, (ii) the methylation status of ≥5 million CpG sites is determined, and (iii) the method includes no more than 9 amplification cycles (e.g. no more than 9 PCR amplification cycles). In some embodiments, the methods cover >2 million CpG sites at a depth >400.

In one embodiment, a method according to the invention comprises the following steps:
(a) completely digesting cfDNA extracted from ≤10 ml human blood with restriction endonuclease(s), comprising a plurality of methylation-sensitive restriction endonucleases;
(b) amplifying the digested DNA, wherein the amplification comprises no more than 9 amplification cycles (e.g. by PCR, using no more than 9 PCR cycles);
(c) sequencing the amplified DNA by a next-generation sequencing method to provide sequencing data with an average depth of ≥600 across the genome; and
(d) determining from the sequencing data the methylation level of ≥5 million CpG sites, optionally wherein >2 million CpG sites are covered at a depth >400.

The above embodiment is provided merely to illustrate specific aspects of the invention, which is described in more detail below.

### Interrogation of the Whole Human Genome

Methods of the invention can interrogate the whole genome, covering ≥5 million CpG sites, using cfDNA from ≤10 ml blood. Methods of the invention may therefore not include a step in which particular target sites within the human genome are enriched. Rather, cfDNA from across substantially the whole human genome can be sequenced.

The term ‘CpG site’ means a cytosine in the context of a CG dinucleotide sequence and is also known as a ‘CpG dinucleotide’. As there are approximately 28 million CpG sites in the haploid human genome, the methods can interrogate at least around 20% of the total number of sites. It is not possible to achieve such coverage using techniques available in the prior art, such as those based on bisulfite sequencing, without using much larger quantities of blood or many more amplification cycles, both of which are undesirable. Thus, the ability to achieve coverage of at least around 20% of genomic CpG sites is one of the advantages of the methods of the invention.

Interrogating the whole human genome at ≥5 million CpG sites means determining a methylation level and/or unmethylation level for each of these CpG sites.

In some embodiments, the methods achieve even greater coverage of CpG sites. Thus, in some embodiments, the number of CpG sites interrogated is at least 5.5, 6, 7, 8, 9, or 10 million, or more.

The methylation level of a CpG site is a numerical value that represents the number or proportion of molecules in which the CpG site is methylated, compared to the total number of molecules in the sample which contain that CpG site. Conversely, the unmethylation level of a CpG site is a numerical value that represents the number or proportion of molecules in which the CpG site is not methylated, compared to the total number of molecules in the sample which contain the CpG site.

Advantageously, the methods of the invention allow the methylation level and unmethylation level of CpG sites to be determined independently from each other but using the same sequencing data from a single assay. The ability to directly determine these complementary methylation indicators provides for improved methylation profiling and increased sensitivity.

CpG sites are not randomly distributed throughout eukaryotic genomes and are frequently found in clusters known as ‘CpG islands’. These islands have been formally defined (Gardiner-Garden & Frommer (1987) J Mol Biol 196:261-82) as regions which are at least 200 bp long, having 50% or more GC content, and where the observed-to-expected CpG ratio is greater than 60% (i.e. where the number of CpG sites multiplied by the length of the sequence, divided by the number of C multiplied by the number of G, is greater than 0.6). CpG islands are often found near the start of a gene in mammalian genomes, and about 70% of promoters near transcription start sites in the human genome contain a CpG island. Methylation of multiple CpG sites within a promoter's CpG island is generally associated with stable silencing of gene expression from that promoter.

The human genome sequence contains around 28 million CpG sites (per haploid genome), with around 30,000 CpG islands. In any particular nucleated cell, some CpG sites will be methylated and others will not. Patterns of methylation can differ between different cells and tissues within a subject, such that a specific CpG can be methylated in one cell or tissue but unmethylated in a different cell or tissue within the same subject.

It is known that tumors can display different methylation patterns compared to non-tumor cells (or compared to other types of tumor). Some sites can become hypermethylated in tumors, while others can become hypomethylated, and the difference in these patterns has been used to aid tumor diagnosis.

### Average Depth

Methods of the invention provide an average sequencing depth of >600 across the human genome. As used herein, “depth” refers to the number of times a particular genomic locus is spanned by sequence reads produced in the high throughput sequencing step. As explained below, “depth” also refers to the number of indirect assessments of the methylation status of a particular genomic locus, made by inference based on sequence reads from the same molecule mapping to upstream and downstream of a locus. Hence, depth can be assessed directly, from sequence reads which terminate at or span a locus, or indirectly from upstream and downstream reads of the same molecule. For a particular locus, a depth of 600 indicates 600 assessments of the methylation status of the locus, including both direct and indirect assessments.

The term “average” refers to the arithmetic mean. Thus, the average depth can be calculated by dividing the sum of nucleotides in sequence reads which map to the human genome by the length of the (haploid) human genome.

Moreover, the methods can cover >2 million CpG sites at a depth >400 i.e. the sequence reads permit the methylation status of at least 2 million CpG sites to be assessed more than 400 times each. This can mean that there are at least 400 sequence reads which terminate at or span these CpG sites, but the methylation status of a CpG site can also be assessed if the termini of sequence reads for an individual molecule map upstream and downstream of the CpG site because it can then be inferred that the CpG was undigested in between these termini even though the CpG sequence was not directly observed.

Higher sequencing depth increases accuracy by allowing signal to be better distinguished from noise. This is because high-throughput DNA sequencing is error prone (with approximately 0.1-10% of all called bases being incorrect), so sequence reads mapped to a particular locus often will often contain mutations compared to the reference sequence at that site and/or the other reads mapped to that site.

High depth is useful for determining whether differences in the sequence reads with respect to the reference sequence reflect the underlying sequence of the sample DNA (signal) or are due to errors during sequencing (noise). High depth therefore aids in drawing meaningful conclusions from sequencing data, particularly regarding the presence of rare signals, such as those that result from tumor DNA. Methylated cfDNA molecules from a tumor may be present in blood plasma at amounts in the order of ≤1% of the total cfDNA. Bisulfite sequencing does not provide sufficient depth across a large enough number of genomic sites to reliably detect these rare signals. In contrast, the methods of the invention interrogate CpG sites in the whole human genome with very high average depths, so allowing the detection of these rare signals.

In some embodiments, the average depth is ≥625, ≥650, ≥675, ≥700, ≥725, ≥750, ≥775 or ≥800. Prior art methods cannot achieve such high sequencing depths with only the small amounts of cfDNA available from 10 ml of blood without using more than 9 cycles of PCR amplification.

Both strands of a starting cfDNA molecule may be read independently, and both reads contribute to the calculation of depth.

Any particular CpG site can give rise to different types of sequencing read depending on whether it is methylated or unmethylated. For instance, if a MSRE is used and a particular CpG is methylated then the cfDNA molecule will not be cleaved and the intact restriction site will be seen in a sequencing read. Conversely, if the CpG is unmethylated then the molecule will be cleaved and reads of the molecule will start or end with the sequence resulting from the cleavage. When a molecule is cleaved, thereby providing two molecules, these two molecules are together counted only once when assessing sequencing depth. To avoid double-counting, therefore, the non-methylated CpG sites (when using a MSRE) can be taken as sequencing reads whose 5′ ends map to a site, as sequencing reads whose 3′ ends map to a site, or as the half of the sum of sequencing reads whose 5′ ends or 3′ ends map to a site. As some library preparation methods can result in depletion of small fragments, which are then not sequenced (e.g. in CpG islands, where a starting cfDNA molecule is cleaved by a MSRE at more than one unmethylated site, thus providing 3 or more restriction fragments, some of which are very small), the observed number of unmethylated CpG sites may be lower than the true value in the original sample. This distortion can be somewhat addressed by using the larger of the number of reads whose 3′ ends map to a site and the number of reads whose 5′ ends map to a site (or to use the mean).

After restriction digestion it is preferred to use fill-in end repair when preparing a sequencing library, so that the terminal sequences of the cleaved molecules are retained.

### Blood Sample

In a further aspect, the methods of the invention are performed on ≤10 ml blood. In some embodiments, ≤9 ml, ≤8 ml, ≤7 ml, ≤6 ml or ≤5 ml blood is used. In some embodiments, 5-10 ml blood is used.

Blood comprises DNA that is found inside cells and DNA that is freely circulating outside of cells (known as “cell-free DNA”). The methods of the invention interrogate the methylation of cell-free DNA from the blood sample.

The origin of cfDNA is not fully understood, but it is generally believed to be released from cells in processes such as apoptosis and necrosis. cfDNA is highly fragmented compared to intact genomic DNA (e.g. see Alcaide et al. (2020) Scientific Reports 10, article 12564), and in general circulates as fragments between 120-220 bp long, with a peak around 168 bp (in humans).

Preferably, the cfDNA utilised in methods and composition disclosed herein is substantially free of single-stranded DNA (ssDNA) i.e. where less than 7% of the cfDNA molecules (by number) are single-stranded, and preferably less than 5% or less than 1% (i.e. such that at least 99% of the cfDNA molecules are double-stranded). In some embodiments, the cfDNA contains less than 0.1% ssDNA, less than 0.01% ssDNA, or may even contain no ssDNA (i.e. free of ssDNA). Extraction of cfDNA to obtain a cfDNA sample substantially free of ssDNA is described, for example, in WO2020/188561. Ensuring low levels of ssDNA avoids potential inhibition of restriction digestion, and also avoids undesired amplification of ssDNA. Commercial kits are available for quantifying single-stranded DNA in a sample e.g. the Promega QuantiFluor™ kit.

Blood may be treated to yield plasma (i.e. the liquid remaining after a whole blood sample is subjected to a separation process to remove the blood cells, typically involving centrifugation) or serum (i.e. blood plasma without clotting factors such as fibrinogen). Thus the methods disclosed herein can be used as part of so-called liquid biopsy testing. Methods disclosed herein may include a step of purifying cfDNA from a blood sample. This step may comprise a step of preparing plasma or serum from the blood sample and then purifying cfDNA from the plasma or serum. Methods may also include a step of obtaining a blood sample and preparing plasma or serum therefrom, thus providing a source for downstream purification of cfDNA.

Blood can be collected in tubes that contain an anticoagulant and an agent to inhibit genomic DNA from white blood cells in the sample being released into the plasma component of the blood sample. Such tubes are commercially available as glass cfDNA ‘Blood Collection Tubes’ or ‘BCT’ from Streck (La Vista, NE) e.g. as discussed by Diaz et al. (2016) PLOS One 11 (11): e0166354, and they can stabilize cfDNA within blood for up to 14 days at 6-37° C. (thus providing advantages compared to typical K₂EDTA collection tubes). Useful anticoagulants include, but are not limited to, EDTA, heparin, or citrate. Useful agents to inhibit release of genomic DNA from white blood cells include, but are not limited to, diazolidinyl urea, imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxy-methoxymethyl-1-laza-3,7-dioxabicyclo[3.3.0]octane, 5-hydroxymethyl-1-1 aza-3,7dioxa-bicyclo[3.3.0]octane, 5-hydroxypoly[methyleneoxy]methyl-1-laza-3,7dioxabicyclo[3.3.0]-octane, quaternary adamantine, and mixtures thereof. Other useful components can include a quenching agent (e.g. lysine, ethylene diamine, arginine, urea, adenine, guanine, cytosine, thymine, spermidine, or any combination thereof) which can abate free aldehyde from reacting with DNA within a sample, aurintricarboxylic acid, metabolic inhibitors (e.g. glyceraldehyde and/or sodium fluoride), and/or nuclease inhibitors. For instance, a tube can include imidazolidinyl urea (or diazolidinyl urea), EDTA and glycine. Further information about suitable collection tubes can be found in WO2013/123030 and US2010/0184069.

Other useful collection tubes are available, including but not limited to various plastic tubes: the ‘Cell-Free DNA Collection Tube’ from Roche, made of PET; the ‘LBgard blood tube’ from Biomatrica, made from plastic and suitable for up to 8.5 ml of blood; and the ‘PAXgene Blood DNA tube’ from PreAnalytiX or Qiagen. These various tubes are discussed in more detail in Kerachian et al. (2021) Clinical Epigenetics 13,193 and Grölz et al. (2018) Current Pathobiology Reports 6:275-86.

A 10 ml blood sample typically yields between 10-400 ng cfDNA. Methods disclosed herein can be performed on the amount of cfDNA contained in a 10 ml blood sample. Methods disclosed herein may typically use between 10-400 ng e.g. between 10-250 ng or between 10-200 ng.

Analysis of plasma-derived cfDNA is preferred. Kits for purifying cfDNA from plasma (and other bodily fluids) are readily available e.g. the MagMAX cfDNA isolation kit from ThermoFisher, the Maxwell RSC ccfDNA plasma kit from Promega, the Apostle MiniMax high efficiency isolation kit from Beckman Coulter, or the QIAamp or EZI products from Qiagen.

Methods disclosed herein may therefore utilise cfDNA extracted from ≤10 ml blood from a subject. Methods may begin with cfDNA which has already been prepared, or may include an upstream step of preparing the cfDNA. Similarly, methods may include an upstream step of obtaining a plasma sample before a step of preparing cfDNA from the plasma sample.

In some embodiments, all extracted cfDNA is used in the methods disclosed herein. In other embodiments, cfDNA is split into multiple fractions, and one or more fractions is not used in the methods disclosed herein but may instead be used in other analytical methods, or is kept for use in control experiments, or for other purposes.

In some embodiments, cfDNA is quantified prior to digestion. In other embodiments, cfDNA is not quantified prior to digestion.

### DNA Digestion

The methods of the invention may utilise restriction endonuclease(s), which recognise specific sequences in double-stranded DNA and introduce a double-stranded break into the DNA. More specifically, methylation-sensitive restriction endonucleases (MSREs) and/or methylation-dependent restriction endonucleases (MDREs) may be used to interrogate CpG sites. A MSRE cleaves the target DNA only if a CpG associated with its recognition site is unmethylated, and methylation inhibits the cleavage. Conversely, a MDRE cleaves the target DNA only if a CpG associated with its recognition site is methylated. Type II restriction endonucleases are particularly useful i.e. enzymes where the double-stranded break is introduced within the recognition site.

In embodiments comprising digestion with MSRE(s) and/or MDRE(s), the CpG sites interrogated are associated with the recognition site(s) of the restriction endonuclease(s) used. Recognition sites are also called ‘restriction sites’ or ‘restriction loci’. A CpG site is associated with a restriction locus if it is in or overlaps the locus.

In some embodiments, cfDNA from the sample is digested with MSRE(s). In some embodiments, cfDNA from the sample is digested with MDRE(s).

Enzymes and cfDNA are typically incubated for a long enough period for substantially complete digestion to occur i.e. further incubation does not lead to any measurable increase in cfDNA cleavage. For a typical sample, this can be achieved by incubation at 37° C. for 2 hours, but longer digestions can be performed if desired e.g. 3 hours, 4 hours, or longer (e.g. overnight). In some embodiments, digestion is performed for 11 hours or less e.g. for between 2-10 hours, 2-9 hours, 2-8 hours, or 2-4 hours. In other embodiments (e.g. where a collection tube is used, as discussed herein) digestion may be performed for longer periods e.g. for 12 hours or more.

Allowing a digestion reaction to substantially proceed to completion provides information about the cleavability of the restriction loci of the restriction endonuclease(s) used in the reaction. For example, if a particular restriction locus in a particular DNA molecule is not cleaved after complete digestion, then it can be inferred that the locus in that molecule was not cleavable. Therefore, if the locus was the restriction site of a MSRE, then lack of cleavage indicates that the CpG site associated with the locus in that DNA molecule was methylated, while cleavage indicates that the CpG site associated with the locus in that DNA molecule was unmethylated. Conversely, if the locus was the restriction site of a MDRE, then lack of cleavage indicates that the CpG site associated with the locus in that DNA molecule was unmethylated, while cleavage indicates that the CpG site associated with the locus in that DNA molecule was methylated.

In embodiments comprising digestion of cfDNA, both CpG methylation and CpG unmethylation can be directly and independently determined. For instance, if cfDNA from a sample is completely digested with a MSRE, then the number of digested cfDNA molecules containing a particular un-cleaved restriction locus indicates the number of cfDNA molecules in the sample in which the CpG associated with that locus was methylated. Additionally, the number of times that restriction locus was cleaved indicates the number of cfDNA molecules in the sample in which the CpG associated with that locus was unmethylated. Similar reasoning can be followed for the cases of digestion with a MDRE. As a result, in some embodiments, unmethylation of CpG sites can be determined independently from methylation of CpG sites, but using the same data from a single assay. This enables an improved identification of methylation changes because it provides complementary methylation information, so allowing more accurate and valid assessment of potential DNA methylation markers, and better detection of methylation differences between samples. It also provides an increased sensitivity of methylation analysis, particularly beneficial for genomic regions with extremely high or extremely low methylation levels.

MSREs and MDREs are readily available from well-known commercial suppliers, such as ThermoFisher, New England Biolabs, Promega, etc.

MSREs include, but are not limited to: AatII, AccII, AciI, AclI, AfeI, AgeI, Aor13HI, Aor51HI, AscI, AsiSI, AvaI, BceAI, BmgBI, BsaAI, BsaHI, BsiEI, BsiWI, BsmBI, BspDI, BspT104I, BssHII, BstBI, BstUI, Cfr10I, ClaI, CpoI, DpnII, EagI, Eco52I, FauI, FseI, FspI, HaeII, HapII, HgaI, HhaI, HinPII, HpaII, Hpy99I, HpyCH4IV, KasI, MluI, MspI, NaeI, NarI, NgoMIV, NotI, NruI, NsbI, PaeR7I, PluTI, PmaCI, PmlI, Psp1406I, PvuI, RsrII, SacII, SaII, ScrFI, SfoI, SgrAI, SmaI, SnaBI, SrfI, TspMI, ZraI, and high-fidelity (HF®) versions of any of these listed enzymes.

MDREs include, but are not limited to: BspEI, BtgZI, FspEI, GlaI, LpnPI, McrBC, MspJI, XhoI, XmaI.

Two preferred MSREs are HinPII and AciI.

As the maximum number of sites that can be interrogated using a MSRE or MDRE is the same as the number of restriction loci present in the human genome for the MSRE/MDRE (or combination thereof) used, the MSRE(s) and/or MDRE(s) are chosen to recognise ≥5 million restriction loci in the human genome. It may be possible to interrogate even greater numbers of CpG sites (e.g. at least 8 million, at least 10 million, at least 15 million, or at least 20 million) by, for instance, using additional MSRE(s) and/or MDRE(s) that recognise additional restriction loci.

The methods disclosed herein can comprise a plurality of restriction endonucleases, wherein the plurality consists of MSRE and/or MDRE. Thus the plurality may include only MSREs, only MDREs, or a mixture of both (e.g. one or more MSRE plus one or more MDRE). In general, however, it is preferred to work with MSREs, without needing MDREs, and thus the plurality includes two or more MSREs. Using MSREs leads to digested cfDNA in which methylated CpG sites are intact but unmethylated CpG sites are digested. Thus, for any particular CpG-containing restriction site in a cfDNA sample, a higher percentage of methylation at this site leads to a lower extent of digestion compared to a cfDNA sample containing a higher percentage of methylation at this site.

A preferred plurality of MSREs includes both HinP1I and AciI. In some embodiments it is possible to use one or more MSREs in addition to HinP1I and AciI, but it is more preferred to use HinP1I and AciI as the only two restriction enzymes for digestion of cfDNA. This pairing of enzymes covers over 99% of CpG islands in the human genome. With this MSRE pairing it is preferred to include HinP1I at an excess (measured in terms of enzymatic units) to AciI, and ideally an excess of at least 1.2:1 e.g. at least 1.5:1, at least 1.75:1, at least 2:1, at least 3:1, at least 4:1, or at least 5:1. Ratios between 2:1 and 5:1 are particularly useful with human cfDNA, and an excess of about 4.5 is preferred. Digestion can be performed at about 37° C., until completion. Incubation at 37° C. for 2 hours is typically adequate for complete digestion with HinP1I and AciI.

HinP1I (sometimes known as Hin6I) recognises the sequence GCGC and cleaves after the first G to leave a two nucleotide 5′ overhang (5′-G/CGC). It cuts well at 37° C. and can be heat-inactivated by heating at 65° C. for 20 minutes. For HinP1I, NEB recommends the use of its rCutSmart™ buffer (50 mM potassium acetate, 20 mM Tris-acetate, 10 mM magnesium acetate, 100 μg/mL recombinant albumin, pH 7.9). 1 unit of HinP1I is defined as the amount of enzyme required to digest 1 μg of 2 DNA in 1 hour at 37° C. in a total reaction volume of 50 μl.

AciI recognises the sequence CCGC and cleaves after the first C to leave a two nucleotide 5′ overhang (5′-C/CGC). It cuts well at 37° C. and can be heat-inactivated by heating at 65° C. for 20 minutes. For AciI, NEB recommends the use of its rCutSmart™ buffer (50 mM potassium acetate, 20 mM Tris-acetate, 10 mM magnesium acetate, 100 μg/mL recombinant albumin, pH 7.9). 1 unit of AciI is defined as the amount of enzyme required to digest 1 μg of A DNA in 1 hour at 37° C. in a total reaction volume of 50 μl. Its recognition site is non-palindromic.

λ DNA is a commonly used DNA substrate extracted from bacteriophage lambda (cI857ind 1 Sam 7), being 48502 bp long. It is usually stored in 10 mM Tris-HCl (pH 8.0), 1 mM EDTA, and is widely available from commercial suppliers e.g. from NEB under catalogue number N3011S.

After digestion has occurred, it is preferred to inactivate the restriction enzymes, particularly if downstream amplification steps will be used. HinP1I and AciI can both be inactivated by heating at 65° C. In some embodiments heating at this temperate occurs for longer than 15 minutes, and ideally occurs for at least 20 minutes e.g. for 20-60 minutes. The temperature can exceed 65° C. if desired, but this is not required. This heating step is adequate for complete inactivation of the restriction enzymes i.e. such that the enzymes' digestion activity which was present during cfDNA digestion can no longer be measurably detected even when cleavable target molecules are present.

Preferred methods do not include a step of bisulfite conversion. Other preferred methods include no step in which chemical changes are made to nucleobases within DNA e.g. no bisulfite conversion, no TAPS conversion, etc. TAPS conversion refers to TET-assisted pyridine borane sequencing.

Preferred methods do not use restriction enzyme isoschizomers, where one of the enzymes recognizes both the methylated and unmethylated forms of the restriction site while the other recognizes only one of these forms.

Preferred methods do not use a mixture of restriction enzymes in which at least one enzyme has a recognition sequence which includes a CpG but which is neither a MSRE or a MDRE i.e. an enzyme which digests regardless of the CpG methylation status.

Where methods are described herein as involving “digestion”, this term (and also “digesting”, etc.) refers to the mixing of active restriction enzyme(s) with cfDNA in conditions under which digestion can occur. If there are no recognition sites for the restriction enzyme in question (e.g. because it is a MSRE and all of the recognition sequences are fully methylated) then a step of “digestion” still takes place even though DNA cleavage does not occur.

### Noise

Methods for interrogating the whole human genome of the invention are associated with lower levels of noise than the genome interrogation methods of the prior art. The term “noise” refers to changes occurring in the sample as the method is performed which affect the output data. Values for noise are given as percentage values, reflecting the percentage of data values affected by the changes.

Noise can take the form of false methylation. In embodiments involving MSRE digestion, false methylation noise may be due to incomplete digestion because lack of cleavage of a locus due to incomplete digestion is indistinguishable from lack of cleavage due to CpG site methylation. In embodiments involving digestion with methylation-dependent endonucleases, false unmethylation noise may be due to digestion of an unmethylated restriction locus (i.e. one containing or overlapping with an unmethylated CpG site) because a cleaved locus would be assumed to be methylated.

Noise can also take the form of false unmethylation. In embodiments involving MSRE digestion, false unmethylation noise may be due to digestion of a methylated restriction locus (i.e. one containing or overlapping with a methylated CpG site). In embodiments involving digestion with methylation-dependent endonucleases, false unmethylation noise may be due to incomplete digestion.

Prior art genome interrogation methods are associated with much higher levels of false methylation noise and false unmethylation noise than the methods of the invention. For example, bisulfite sequencing is associated with false methylation and false unmethylation noise levels of about 1%. This means that in genome interrogation methods based on bisulfite sequencing, at least about 1% of the sites interrogated will be wrongly determined to be methylated or unmethylated. False methylation noise in bisulfite sequencing may result from incomplete bisulfite conversion of unmethylated cytosine to uracil which can be due to various causes, such as impurities in the sample DNA. False unmethylation noise results from over-conversion of 5mC.

In contrast, the genome interrogation methods of the invention are associated with very low levels of noise, both for false methylation and false unmethylation. In some embodiments, the level of false methylation/unmethylation noise is ≤0.05%, 0.04%, 0.03%, 0.02%, 0.01% or 0.00%. In preferred embodiments, the level of false methylation noise is ≤0.03% and the level of false unmethylation noise is 0.00%.

### PCR

Only a small amount of cfDNA is available from 10 ml blood (e.g. typically between 10-400 ng). Further, only a very small fraction of this cfDNA may be derived from tumor cells. Therefore, methods disclosed herein may include a step of amplification (e.g. by PCR), but they use no more than 9 amplification cycles (e.g. no more than 9 PCR cycles). Amplification may be performed on digested cfDNA, and may occur during library preparation for next-generation sequencing, using a set of primers directed to sequences within the sequencing adapters (see below).

No more than 9 amplification cycles are used, and some embodiments involve no more than 8, 7, 6, 5, 4, 3, 2 or 1 amplification cycles. Some embodiments involve no amplification at all.

Typically, any amplification takes place during a library preparation step for a next-generation sequencing reaction (see below). Thus, preferred methods do not involve any amplification cycles prior to sequencing library preparation.

Using as few amplification cycles as possible is desirable because each cycle introduces noise into the data. Firstly, the polymerase enzymes used in PCR will preferentially amplify DNA molecules with certain characteristics. This results in copy number biases in amplified DNA as the preferentially amplified DNA molecules will be over-represented in the amplified DNA compared to the DNA before amplification. Also, PCR amplification will introduce mutations into the amplified DNA because the fidelity of DNA polymerases is not 100%.

By keeping the number of amplification cycles involved in the methods of the invention to no more than 9, the noise resulting from amplification is minimised. The inventor has surprisingly discovered that it is possible to obtain interpretable signal for the methylation level of ≥5 million CpG sites with cfDNA from ≤10 ml blood even with no more than 9 amplification cycles. If only the techniques available in the prior art are used, then many more than 9 amplification cycles are required to generate interpretable signal with cfDNA from ≤10 ml of blood, because of the higher levels of noise associated with these prior art techniques.

Methods may therefore include a step of adding amplification reagents e.g. suitable buffer/salt components (if required in addition to buffer/salt remaining from digestion), a DNA polymerase (such as a Taq polymerase), dNTPs, primers and (optionally) probes.

Restriction digestion typically takes place in the presence of high levels of Mg⁺⁺. PCR usually relies on Mg⁺⁺, so standard PCR buffers include Mg⁺⁺. In this situation, however, addition of a standard PCR buffer can lead to an excess of Mg⁺⁺ which can inhibit efficiency of amplification. Thus, in methods involving restriction digestion, added PCR reagents may include a lower level of Mg⁺⁺ than would normally be the case.

Amplification primers may vary in length, depending on the particular assay format and the particular needs. In some embodiments, the primers may be at least 15 nucleotides long, such as between 15-25 nucleotides or 18-25 nucleotides long. The primers may be adapted to be suited to a chosen amplification system.

Primers may be designed to generate amplicons between 60-150 bp long (when the relevant CpG site(s) is/are intact) e.g. between 70-140 bp long.

Computer software is readily available for routine designing of primers and probes which meet the various requirements of any particular experiment.

### High Throughput Sequencing

The methods disclosed herein include a step of DNA sequencing using high throughput sequencing techniques (also known as next generation sequencing, or NGS). High throughput sequencing generally involves three basic steps: library preparation; sequencing; and data processing. Examples of high throughput sequencing techniques include sequencing-by-synthesis and sequencing-by-ligation (employed, for example, by Illumina Inc., Life Technologies Inc., PacBio, and Roche), nanopore sequencing methods and electronic detection-based methods such as Ion Torrent™ technology (Life Technologies Inc.). High throughput sequencing may be performed using various commercially available sequencing instruments and platforms, including but not limited to: Novaseq™, Nextseq™ and MiSeq™ (Illumina), 454 Sequencing (Roche), Ion Chef™ (ThermoFisher), SOLID® (ThermoFisher) and Sequel II™ (Pacific Biosciences). Appropriate platform-designed sequencing adapters are used for preparing the sequencing library, and are readily available from the platforms' manufacturers.

Library preparation for the major high-throughput sequencing platforms involves ligation of specific adapter oligonucleotides, also termed ‘sequencing adapters’, to the DNA fragments to be sequenced. Sequencing adapters typically include platform-specific sequences for fragment recognition by a particular sequencer e.g. sequences that enable ligated molecules to bind to the flow cells of Illumina platforms (e.g. the P5 and P7 sequences). Each sequencing instrument provider typically sells a specific set of sequences for this purpose. Further details of library preparation are discussed below.

Sequencing adapters can include sites for binding to a universal set of PCR primers. This permits multiple adapter-ligated DNA molecules to be amplified in parallel by PCR, using a single set of primers.

Sequencing adapters can include sample indices, which are sequences that enable multiple samples to be combined, and then sequenced together (i.e. multiplexed) on the same instrument flow cell or chip. Each sample index, typically 6-10 nucleotides, is specific to a given sample and is used for de-multiplexing during downstream data analysis to assign individual sequence reads to the correct sample. Sequencing adapters may contain single or dual sample indexes depending on the number of libraries combined and the level of accuracy desired.

Sequencing adapters can include unique molecular identifiers (UMIs) to provide molecular tracking, error correction and increased accuracy during sequencing. UMIs are short sequences, typically 5 to 20 bases in length, used to uniquely identify original molecules in a sample library. As each nucleic acid in the starting material is tagged to provide a unique molecular barcode, bioinformatics software can filter out duplicate reads and PCR errors with a high level of accuracy and report unique reads, removing the identified errors before final data analysis.

In some embodiments, sequencing adapters include both a sample barcode sequence and a UMI.

In some embodiments, sequencing adapters allow for paired-end sequencing.

In some embodiments, the methods disclosed herein use Y-shaped sequencing adapters i.e. adapters consisting of two single-stranded oligonucleotides which anneal to provide a double-stranded stem and two single-stranded ‘arms’. In other embodiments, methods disclosed herein use hairpin sequencing adapters i.e. a single-stranded oligonucleotide whose 5′ and 3′ termini anneal to provide a double-stranded stem. For both Y-shaped and hairpin adapters the double-stranded stem can include a short single-stranded overhang e.g. a single A or T nucleotide. For both Y-shaped and hairpin adapters the double-stranded stem can be ligated to a cfDNA fragment, to prepare a sequencing library.

Suitable sequencing adapters for use in the methods disclosed herein may thus be TruSeq™ or AmpliSeq™ or TruSight™ adapters (for use on the Illumina platform) or SMRTbell™ adapters (for use on the PacBio platform).

Where sequencing adapters are added by ligation, this usually occurs at both ends of the DNA to be sequenced.

Restriction digestion can leave blunt-ends, but typically produces a single-stranded overhang. Library preparation steps can either preserve this overhang (i.e. add complementary nucleotides) or remove it. As the sequence of a post-digestion terminal single-stranded overhang can include useful information then it is preferred to add sequencing adapters in a way which preserves the overhang e.g. using enzymatic ligation in which a ligase enzyme covalently links a sequencing adapter to a DNA fragment where the terminal sequence of the adapter is complementary to the terminal sequence obtained using the restriction enzyme, or by using a polymerase to add complementary nucleotides and generate a blunt-ended fragment.

In addition to removing or filling in single-strand overhangs, end repair methods can be carried out before adapter ligation can ensure that DNA molecules contain 5′ phosphate and 3′ hydroxyl groups.

For some libraries, incorporation of a non-templated deoxyadenosine 5′-monophosphate (dAMP) onto the 3′ end of blunted DNA fragments is used in library preparation (a process known as dA-tailing). dA-tails prevent concatemer formation during downstream ligation steps and enable DNA fragments to be ligated to adapter oligonucleotides with complementary dT-overhangs.

As noted above, restriction digestion typically takes place in the presence of high levels of Mg⁺⁺. Sequencing library preparation may also rely on Mg⁺⁺, so standard library prep buffers include Mg⁺⁺. In this situation, however, addition of a standard library prep buffer can lead to an excess of Mg⁺⁺ which can inhibit efficiency of downstream steps. Thus added reagents may include a lower level of Mg⁺⁺ than would normally be the case for library preparation.

After library preparation, the prepared DNA molecules can be sequenced, to provide a plurality of ‘sequence reads’. These sequence reads are then subjected to data processing e.g. to remove sequences which do not fulfil desired quality criteria, to remove duplicates, to correct sequencing errors, to map sequences onto a reference genome, to count the number of sequence reads, etc. Computer software is readily available for performing these steps. This is described in more detail below.

The methods disclosed herein do not require differential adapter tagging of methylated vs. unmethylated DNA molecules. The same population of adapters are used for the entire sample.

### Analysis of Sequence Reads

Any particular CpG site can feature in multiple sequence reads, which can be sequence reads derived from the same original cfDNA molecule and/or from different cfDNA molecules which span the same CpG site.

Sequence reads can be mapped to a reference genome i.e. a previously identified genome sequence, whether partial or complete, assembled as a representative example of a species or subject. A reference genome is typically haploid, and typically does not represent the genome of a single individual of the species but rather is a mosaic of the genomes of several individuals. A reference genome for the methods of the present invention is typically a human reference genome e.g. a complete human genome, such as the human genome assemblies available at the website of the National Center for Biotechnology Information or at the University of California, Santa Cruz, Genome Browser. An example of a suitable reference genome for human studies is the ‘hg18’ genome assembly. As an alternative, the more recent GRCh38 major assembly can be used (up to patch p13).

Mapping aligns sequence reads to the reference genome, to identify the location of the reads within the reference genome. The sequence reads that align are designated as being ‘mapped’. The alignment process aims to maximize the possibility for obtaining regions of sequence identity across the various sequences in the alignment, allowing mismatches, indels and/or clipping of some short fragments on the two ends of the reads. The number of sequence reads mapped to a certain genomic locus is referred to as the ‘read count’ or ‘copy number’ of this genomic locus. It is not necessary to map all sequence reads which are obtained; indeed, it is not unusual that a portion of sequence reads obtained in any given experiment will not be mappable.

The term ‘genomic locus’ refers to a specific location within the genome, and may include a single position (a single nucleotide at a defined position in the genome) or a stretch of nucleotides starting and ending at defined positions in the genome. The specific position(s) may be identified by the molecular location, namely, by the chromosome and the numbers of the starting and ending base pairs on the chromosome. A genomic locus of interest herein contains at least one CpG site.

The methylation level of a CpG site is the methylation level of the restriction locus associated with the CpG site. In some embodiments, this methylation level is calculated by dividing the read count of the restriction locus, or the read count of a predefined genomic region of at least 50 bp that contains the restriction locus, by an expected read count of the restriction locus or the predefined genomic region of at least 50 bp that contains the restriction locus. An expected read count of the restriction locus/predefined genomic region may be determined, for example, using: (i) read count of a reference locus/genomic region of the same length as the restriction locus containing the restriction locus/genomic region, that is not cut by the restriction endonuclease; (ii) average read count of a plurality of reference loci/genomic regions of the same length as the restriction locus of/genomic region, that are not cut by the restriction endonuclease; or (iii) read count of the restriction locus/predefined genomic region in an undigested control DNA sample, optionally corrected for sequencing depth differences.

In some embodiments, the predefined genomic region is at least 60 bp, 70 bp, 80 bp, 90 bp or 100 bp. In some embodiments, the predefined genomic region is between 50-150 bp, between 50-120 bp or between 50-100 bp. In preferred embodiments, the predefined genomic region is at least 100 bp.

Additionally or alternatively, the methylation level of a CpG site may be calculated by dividing the read count of the restriction locus, or the read count of a predefined genomic region of at least 50 bp that contains the restriction locus, by a total fragment number. The total fragment number is the sum of the read count of the restriction locus associated with the CpG site and the read count of reads whose termini map to this CpG site, taking account where necessary of any end-repair which took place during library preparation.

To avoid double-counting, the cleaved CpG sites can be taken as sequencing reads whose 5′ ends map to a site, as sequencing reads whose 3′ ends map to a site, or as the half of the sum of sequencing reads whose 5′ ends or 3′ ends map to a site. As some library preparation methods can result in depletion of small fragments, which are then not sequenced (e.g. in CpG islands, where a starting cfDNA molecule is cleaved by a MSRE at more than one unmethylated site, thus providing 3 or more restriction fragments, some of which are very small), the observed number of unmethylated CpG sites may be lower than the true value in the original sample. This distortion can be somewhat addressed by using the larger of the number of reads whose 3′ ends map to a site and the number of reads whose 5′ ends map to a site (or to use the mean).

These calculations can thus provide, for any given CpG site, the methylation level of the CpG site. Conversely, similar calculations can provide the unmethylation level of a CpG site. These figures can be expressed as a percentage, a fraction, a normalised value, etc.

### Controls

Methods disclosed herein can take advantage of positive and negative controls. In some embodiments, parallel analysis can be performed on one or more of:
A DNA control which does not contain a recognition sequence for the restriction enzymes used for digestion. If this DNA is digested, this indicates that the method has not performed correctly.
A DNA control which contains a fully methylated recognition sequence for the restriction enzymes used for digestion. If this DNA is digested when a method uses only MSREs, this indicates that the method has not performed correctly (and conversely for MDREs).
A DNA control which contains a fully unmethylated recognition sequence for the restriction enzymes used for digestion. If this DNA is not fully digested when a method uses only MSREs, this indicates that the method has not performed correctly (and conversely for MDREs).

These DNA controls can also be used as a reference point for analysis, for checking completeness of digestion, etc. As mentioned above, for instance, if fragments are obtained using MSRE digestion then it can be useful in a downstream NGS experiment to know the expected read count, and one way of obtaining this value is to look at the read count for DNA which does not contain the recognition sequence for the MSRE, or at the read count for DNA which contains the recognition sequence but is fully methylated.

For these purposes, it is preferred that the DNA control should be similar in size and composition to cfDNA molecules which contain CpG sites of interest. Thus, although it is possible to use synthetic DNA or PCR amplicons or bacterial plasmid DNA as an unmethylated control, these are more useful if they have sizes which are similar to cfDNA (e.g. a long synthetic DNA, or an appropriately-sized restriction fragment prepared from a plasmid).

Control experiments can be performed internally in a sample, or externally. For an internal control, control DNA can be present in a sample already (e.g. cfDNA containing a CpG site which is known to be ubiquitously (un)methylated, or cfDNA which does not contain a recognition sequence for the restriction enzymes being used) and/or can be added (e.g. synthetic DNA, added to cfDNA). The control DNA can therefore be processed in combination with the cfDNA, and experiences the same conditions as the cfDNA, and so a method can involve co-amplification of a restriction locus and a control locus. For an external control, control DNA is subjected to the same treatment as the cfDNA but not as part of the same reaction mixture.

Thus control DNA, like cfDNA, can be digested with restriction enzymes and then subjected to downstream analytical steps e.g. amplification, DNA sequencing, etc. Real-time PCR of suitable control loci can give a result that can be used as a reference point. For instance, the signals obtained from cfDNA at a CpG site of interest and from control DNA (in particular, from control DNA which is not digested by the restriction enzymes being used) can be compared, and the signal ratio can be used to determine the degree of methylation at a CpG site of interest, because the ratio of signal reflects the ratio of methylation. Thus methods disclosed herein can be performed without requiring evaluation of absolute methylation levels at genomic loci, but rather by calculating a signal ratio between the analysed genomic loci and a control. This contrasts with some conventional methods of methylation analysis for distinguishing between tumor-derived and normal DNA, which require determining actual methylation levels at specific genomic loci. The methods disclosed herein can thus eliminate the need for standard curves and/or additional laborious steps involved in determination of absolute methylation levels, thereby offering a simple and cost-effective procedure. An additional advantage when using an internal control is that signal ratios are obtained for loci amplified in the same reaction mixture under the same reaction conditions, which can help to eliminate sources of potential error.

### General

The practice of the present invention will employ, unless otherwise indicated, conventional methods of chemistry, biochemistry, and molecular biology, within the skill of the art. Such techniques are explained fully in the literature. See, e.g., Methods In Enzymology (Academic Press, Inc.), Green & Sambrook (2012) Molecular Cloning: A Laboratory Manual, 4th edition (Cold Spring Harbor Press), Ausubel et al. (eds) Short protocols in molecular biology, 5th edition (Current Protocols), Molecular Biology Techniques: An Intensive Laboratory Course, (Ream & Field, eds., 1998, Academic Press), Wilson and Walker's Principles and Techniques of Biochemistry and Molecular Biology (Hodmann & Clokie, 2018), Basic Molecular Biology & Techniques—Recent Advances: Molecular Biology & Its Technique (Singh et al., 2021), etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

The term “between” with reference to two values includes those two values e.g. the range “between” 10 mg and 20 mg encompasses inter alia 10, 15, and 20 mg.

Unless specifically stated, a method comprising a step of mixing two or more components does not require any specific order of mixing. Thus components can be mixed in any order. Where there are three components then two components can be combined with each other, and then the combination may be combined with the third component, etc.

The various steps of methods may be carried out at the same or different times, in the same or different geographical locations, e.g. countries, and by the same or different people or entities.

### BRIEF DESCRIPTION OF THE DRAWINGS

FIG. 1 shows the correlation of hits in MSRE-digested (left) or bisulfite-converted (right) samples for patient LNG165 (top) or LNG166 (bottom). The y-axis runs from 0-800 and shows hits from the undigested control, and the x-axis runs from 0-1000 and shows the hits in the treated cfDNA.

FIG. 2 shows the number for CpG sites (y-axis) covered at various depths (x-axis) in patient LNG165 (2A) and patient LNG166 (2B).

FIG. 3 shows sequencing noise per nucleotide (x10-7).

FIG. 4 shows the number of detected hypermethylated markers in patients LNG165 and LNG166. The left-hand bar of each pair is the MSRE-based method, and the right-hand bar is bisulfite-converted.

### EXAMPLES

cfDNA was extracted from blood samples (˜8.5 mL) taken from two treatment-naïve non-small cell lung cancer (NSCLC) patients, identified as LNG165 and LNG166, using a QIAamp® Circulating Nucleic Acid Kit. Each extracted cfDNA sample was divided into two aliquots, one subjected to bisulfite conversion and the other subjected to digestion with the methylation-sensitive restriction enzymes HinP1I and AciI. The enzymes were incubated with the cfDNA at 37° C. for at least 2 hours to permit complete digestion to occur. The cfDNA was not subjected to amplification.

The two samples were then subjected to library preparation and sequencing. A sequencing library was prepared from each sample (enzyme-treated, bisulfite-treated) and also from an untreated control sample, using NEBNext Ultra DNA Library Prep Kit for the enzyme-treated and untreated control samples, and ACCEL-NGS® METHYL-SEQ DNA LIBRARY kit for the bisulfite-treated sample. The libraries were subjected to whole-genome next generation sequencing using Illumina NovaSeq 6000 sequencing platform with S4 flow cell. The sequence reads from each sample were mapped against the complete human genome (hg18 genomic build). Library preparation used fewer than 10 amplification cycles after adapters were attached.

Sequencing metrics were as follows for the two patients:

| | LNG165 | | LNG166 | |
|---|---|---|---|---|
| | MSRE | Bisulfite | MSRE | Bisulfite |
| Number of reads | 13,988,252,662 | 6,206,319,156 | 18,649,633,086 | 9,492,198,638 |
| Mapping rate | 96.34% | 78.38% | 97.87% | 79.24% |
| Unique mapping rate | 89.64% | 77.29% | 91.47% | 78.20% |
| Total number of | 12,540,361,228 | 4,797,333,458 | 17,059,522,795 | 7,423,448,896 |
| uniquely mapped reads | | | | |

These results show that for low amounts of DNA the number of reads that are obtained is significantly reduced when using bisulfite treatment vs. MSRE digestion. The number of reads and, importantly, the number of uniquely-mapped reads obtained for bisulfite-treated DNA was less than half the amount obtained for MSRE-digested DNA. In addition, MSRE-digested DNA showed a unique mapping rate of approximately 90%, whereas the unique mapping rate of bisulfite-treated DNA was <80%.

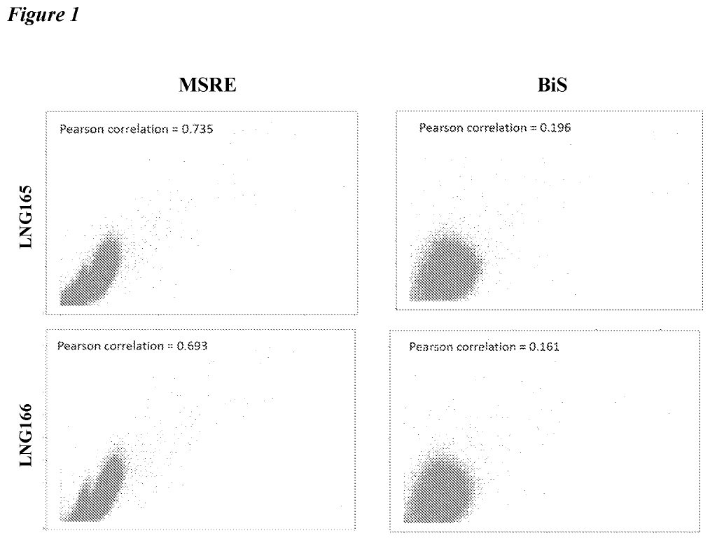

The significant loss of information in the bisulfite-treated sample was further demonstrated by copy number data. Pearson correlation analysis showed correlations of 0.735 and 0.693 in copy number between MSRE-digested DNA and the untreated control sample for the two patients. In contrast, correlations were only 0.196 and 0.161 between bisulfite-treated DNA and the untreated control sample (see FIG. 1). Bisulfite conversion lost nearly all copy number information when low amounts of DNA were used for the assay.

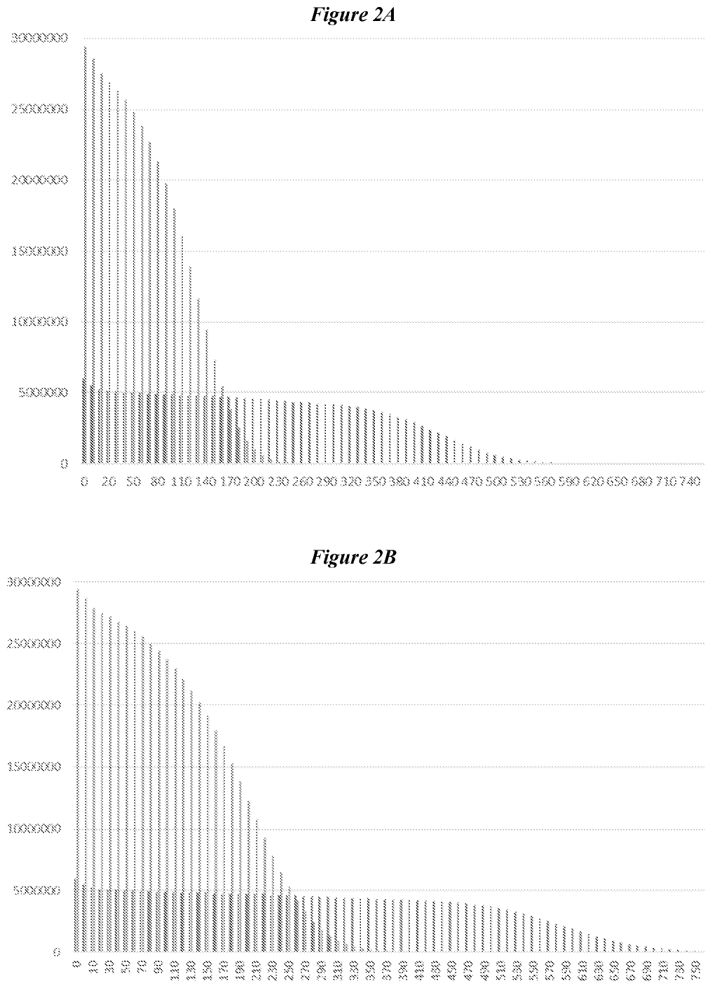

Genome-wide methylation analysis using MSRE digestion is limited to CpG sites which match the recognition sites of the enzyme(s) used in the assay, while bisulfite sequencing in principle covers all CpG sites in the genome. The ability to investigate only a fraction of the CpG sites in the genome has been considered one of the main limitations of restriction enzyme-based methylation analysis. However, FIG. 2 shows that while bisulfite provides broader CpG coverage at lower depths (starting at around 30 million CpG sites in both patients, compared to 6 million using MSRE digestion), a continuous and sharp decrease is seen in the number of CpG sites that are covered in bisulfite-treated DNA as depth increases. In contrast, MSRE digestion shows substantially constant coverage even at depths over 250-300. At high depths, methylation-sensitive digestion provides significantly better CpG coverage compared to bisulfite conversion.

For example, in the DNA sample from patient LNG165 (FIG. 2A), MSRE digestion covered more genomic CpG sites than bisulfite at depths above 165. At a depth of 300, the MSRE-digested sample covered 4.16 million CpG sites, compared to only 44 thousand in the bisulfite-treated sample. In the DNA sample from patient LNG166 (FIG. 2B), MSRE digestion covered more genomic CpG sites at depths above 255, and at a depth of 400 the MSRE-digested sample covered 4.24 million CpG sites, compared to only 65 thousand CpG sites in the bisulfite-treated sample.

MSRE digestion therefore provides coverage of millions of CpG sites at very high depths, enabling the detection of rare methylation signals, as would be seen for an early-stage tumor. The data show that at depths required for identification of rare signals, bisulfite conversion does not provide sufficient coverage, and rare signals are likely to be missed using this technique.

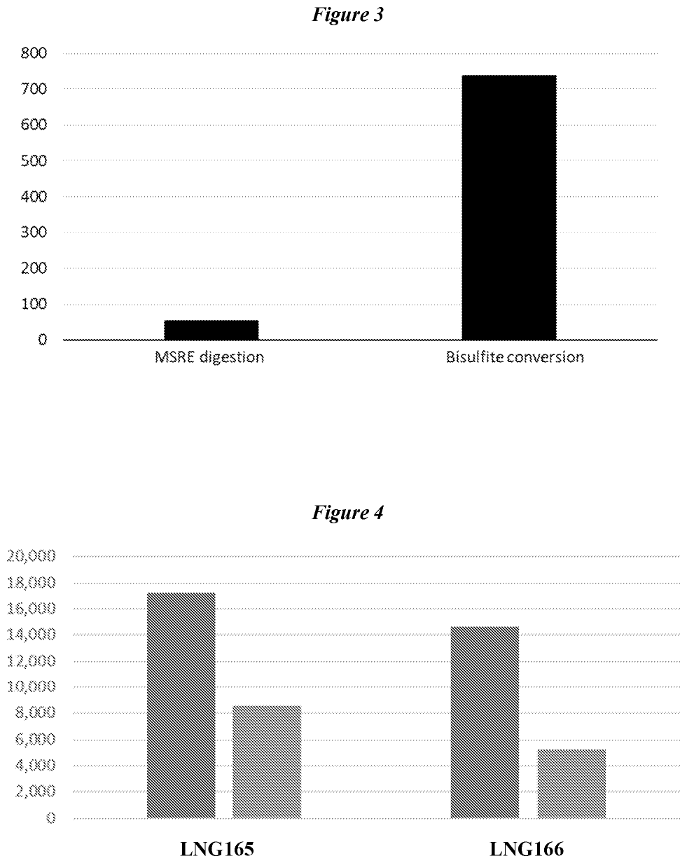

Sequencing noise was assessed for all possible point mutations. In each sample and for each mutation category, 10000 random genomic loci with low background mutation level (i.e., no mutations observed in the plasma of healthy subjects) and with tumor mutation level <0.05 were selected as mutation controls. In these controls, any mutation detected in the plasma of the patient is considered an artifact that is a result of sequencing noise. The mean sequencing noise was calculated, and the results are in FIG. 3. Sequencing noise was 14×lower in MSRE-digested DNA vs. bisulfite treated DNA.

A set of marker loci was compiled which show hypermethylation in tumor vs. normal tissue and are characterized by low background methylation in plasma of healthy individuals. This set of marker loci was compiled based on samples from the two lung cancer patients and a pooled plasma sample of healthy individuals. In addition, a set of isomethylated marker loci, namely, loci which do not show different methylation levels between tumor and normal tissue, was compiled. Methylation levels in cfDNA from each patient was analyzed using MSRE digestion or bisulfite conversion, followed by NGS. A threshold methylation level was set, above which a marker locus was considered as detected. This threshold was based on the set of isomethylated marker loci in order to obtain detection specificity of 95%. The number of marker loci that crossed the detection threshold in the MSRE-digested DNA and in the bisulfite-converted DNA from each patient was compared, and the results are shown in FIG. 4. The MSRE-based method detected significantly more methylation changes in both patients' cfDNA compared to bisulfite conversion.

Tumor mutations were defined as genotypes found in the tumor DNA that are different from the most prevalent genotype in the corresponding normal tissue from the same patient. The fraction of reads with mutated genotypes in the tumor DNA is the tumor mutational level, and the fraction of reads with the same mutated genotypes in cfDNA from a patient represents the plasma mutation level. For each sample, the average tumor and plasma mutation levels were calculated across all mutations and a tumor mutational burden was calculated (i.e., average plasma mutation level/average tumor mutation level). The tumor mutational burden represents the fraction of tumor DNA in the plasma of the patient. To control for sequencing noise, the tumor mutational burden of patient A was compared to a control tumor mutational burden, calculated from the tumor mutations of patient B (i.e., the average mutation level of the tumor mutations of patient B in the plasma of patient A/the tumor mutation level of patient A). Tumor mutations were detected in plasma cfDNA by MSRE-digestion+NGS at levels clearly above sequencing noise, whereas with bisulfite+NGS the mutations were indistinguishable from the high sequencing noise.

It will be understood that the invention has been described by way of example only and modifications may be made whilst remaining within the scope and spirit of the invention.

1. A method for human whole-genome sequencing, comprising:
sequencing human cfDNA from ≤10 ml blood by a high throughput sequencing procedure with an average depth of ≥600 across the genome, wherein the methylation status of ≥5 million CpG sites is determined, and the method includes no more than 9 amplification cycles.

2. The method of claim 1, wherein the amplification cycles are PCR cycles.

3. The method of claim 1, wherein the amplification cycles take place during library preparation for the high throughput sequencing procedure.

4. The method of claim 1, wherein the method provides data with a depth greater than 400 for more than 2 million different CpG sites.

5. The method of claim 1, wherein the number of CpG sites interrogated is at least 5.5 million, 6 million, 7 million, 8 million, 9 million, 10 million, 11 million, or 12 million.

6. The method of claim 1, wherein the average depth across the genome is ≥625, ≥650, ≥675, ≥700 or ≥725.

7. The method of claim 2, wherein the amplification cycles are PCR cycles.

8. The method of claim 2, wherein the amplification cycles take place during library preparation for the high throughput sequencing procedure.

9. The method of claim 2, wherein the high throughput method provides data with a depth greater than 400 for more than 2 million different CpG sites.

10. The method of claim 2, wherein the number of CpG sites interrogated is at least 5.5 million, 6 million, 7 million, 8 million, 9 million, 10 million, 11 million, or 12 million.

11. The method of claim 2, wherein the average depth across the genome is ≥625, ≥650, ≥675, ≥700 or ≥725.
